(19) United States
(12) Patent Application Publication
(10) Pub. No.: US 2012/0232043 A1
(43) Pub. Date: Sep. 13, 2012

(54) LUBIPROSTONE FOR OBSTETRICAL OR GYNECOLOGICAL APPLICATIONS

(75) Inventor: Andre Ulmann, Paris (FR)

(73) Assignee: CEMAG, Paris (FR)

(21) Appl. No.: 13/395,308

(22) PCT Filed: Sep. 15, 2010
(86) PCT No.: PCT/EP10/63547

§ 371 Date: May 16, 2012

Related U.S. Application Data
(60) Provisional application No. 61/242,716, filed on Sep. 15, 2009.

(51) Int. Cl. A61K 31/56; A61P 15/00; A61K 31/352

(52) U.S. Cl. 514/171; 514/456

(57) ABSTRACT

The invention relates to a method for terminating pregnancy or for menstrual induction, which method comprises administering a female subject with lubiprostone, preferably in combination of a progesterone receptor antagonist, such as mifepristone.

### BACKGROUND OF THE INVENTION

First trimester surgical abortion by vacuum aspiration or dilatation and curettage has been the method of choice for early termination of a pregnancy since the 1960s. Medical abortion became an alternative method of first trimester pregnancy termination with the availability of prostaglandins in the early 1970s and anti-progesterones in the 1980s. Various methods for medical termination of pregnancy are now available. The most widely researched drugs are prostaglandins (PGs) alone, mifepristone alone, methotrexate alone, mifepristone with prostaglandins and methotrexate with prostaglandins. For a review, see Kulier et al, Cochrane Database of Systematic Reviews 2004, Issue 1. Art. No.: CD002855. Bygdeman et al, (Contraception. 2003, 68(6):495-8) teach the possibility of using mifepristone, alone or in combination with sulprostone, misoprostol or gemeprost, for medical termination of pregnancy or for menstrual induction, which is a variant of suction aspiration that was originally defined to be performed in women with a menstrual delay of up to 2-3 weeks without knowing if the woman is pregnant or not. A regimen of mifepristone 600 mg followed 2 days later by gemeprost 1 mg vaginally was provided to women with gestational age inferior to 63 days (Rørbye et al, Contraception. 2003 68(4):247-51). From 2001 to March 2006, Planned Parenthood Federation of America (Planned Parenthood) Health centers throughout the United States provided medical abortions principally by a regimen of oral mifepristone, followed 24-48 h later by vaginal misoprostol (Fierstad et al, Contraception, 2009, 80(3):282-6). Combinations of mifepristone and misoprostol are also described in international patent application WO2008/135203. Currently, various doses of mifepristone (most often 200 mg once) followed by misoprostol at various dosages (usually 400 to 1200 μg) or routes (oral, buccal, sublingual, vaginal), or by gemeprost (0.5 or 1 mg vaginally) are the current method for early termination of pregnancy or menstrual induction.

Such methods are still not fully satisfactory. Their effectiveness needs improving and side effects need to be reduced. In particular misoprostol and gemeprost induce side effects such as fever and chills.

### SUMMARY OF THE INVENTION

It is now proposed to use lubiprostone for inducing pregnancy termination, or for menstrual induction, either alone or most preferably in combination with a progesterone receptor antagonist, such as mifepristone.

The invention thus provides a method for terminating pregnancy, or for menstrual induction, which method comprises administering a female subject, with lubiprostone, or a salt or ester thereof.

In a preferred embodiment the method further comprises administering a progesterone receptor antagonist.

Another subject of the invention is a pharmaceutical composition comprising lubiprostone, or a salt or ester thereof, and a progesterone receptor antagonist, in association with a pharmaceutically acceptable carrier.

A still other subject of the invention is a kit comprising:
a. a pharmaceutical composition comprising lubiprostone, or a salt or ester thereof,
b. another pharmaceutical composition comprising a progesterone receptor antagonist, such as mifepristone.

### DETAILED DESCRIPTION OF THE INVENTION

Definitions:

Lubiprostone is 7-[(2R,4aR,5R,7aR)-2-(1,1-difluoropentyl)-2-hydroxy-6-oxo -3,4,4a,5,7,7a-hexahydrocyclopenta[b]pyran-5-yl]heptanoic acid, of formula:

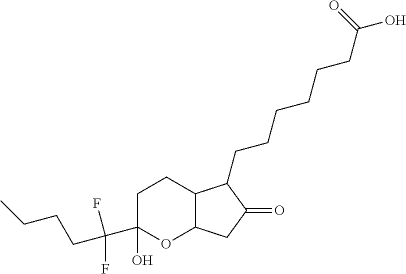

Lubiprostone is a bicyclic fatty acid prostaglandin E 1 derivative that is a locally acting chloride channel activator that enhances a chloride-rich intestinal fluid secretion without altering sodium and potassium concentrations in the serum. Lubiprostone acts by specifically activating CIC-2, which is a normal constituent of the apical membrane of the human intestine, in a protein kinase A-independent fashion. By increasing intestinal fluid secretion, lubiprostone increases motility in the intestine, thereby increasing the passage of stool and alleviating symptoms associated with chronic idiopathic constipation. The use of lubiprostone in chronic idiopathic constipation has been disclosed in U.S. Pat. No. 5,317,032. It is marketed under the trade name Amitiza®. Lubiprostone has mainly a digestive action and is thus expected to be devoid of side-effects on other organs. In the context of the invention, Lubiprostone may be in the form of pharmaceutically acceptable salts, esters, optically active isomer, racemate or hydrates.

As used herein, “female” refers to any animal capable of conception. As such, female includes human and non-human mammals, such as, but not limited to, female domestic and farm animals, zoo animals, sports animals, and pets. In a preferred embodiment, the term female refers to a woman, including a female of pubertal age, i.e., a female between about 12 years old and 17 years old; or an adult female, i.e., a female of about 18 years old or older.

The term “pharmaceutically effective amount” refers to a dosage that provides termination of pregnancy or menstrual induction. Preferably the dosage does not cause excessive toxicity, irritation, allergic response, or other possible complications commensurate with a reasonable benefit/risk ratio.

Indications:

The invention provides methods for inducing pregnancy termination or for menstrual induction, especially in a human female subject. First, second and third trimester pregnancies are encompassed. Preferably the female subject is less than three month pregnant, preferably less with a gestational age of less than 63 days. Termination of second and third trimester pregnancy may also be needed, for instance in cases of congenital or genetic abnormalities, and for the induction of labour in cases of intra-uterine foetal death. The methods of the invention are particularly useful for indications such as induced medical abortion, treatment of “missed” abortion or incomplete miscarriage and dilatation of the cervix prior to transcervical surgical interventions like placing an intra-uterine device (IUD) or a hysteroscope, curettage in pregnant or non-pregnant women and surgical abortion. Menstrual induction is intended for women with a menstrual delay, generally of up to 2-3 weeks, without knowing if the woman is pregnant or not.

Combinations:

Preferably lubiprostone is combined with a progesterone receptor antagonist, either sequentially or within a single pharmaceutical composition.

Mifepristone was the first clinically available progesterone receptor antagonist. Other progesterone receptor antagonists are now available.

In the context of the invention, the term “progesterone receptor antagonist”, also called “antiprogestin” refers to a compound that inhibits the activity of the progesterone receptor. It may be a steroidal or non-steroidal progesterone receptor antagonist or a selective progesterone receptor modulator (SPRM). A SPRM represents a class of progesterone receptor ligands that exerts clinically relevant, tissue selective, mixed progesterone agonist and antagonist effects, which may be full or partial on various progesterone target tissues in an in vivo situation depending on the biological action studied. In the context of the invention, they are classified under the general term “progesterone receptor antagonists”.

Examples of progesterone receptor antagonists and SPRMs include, without limitation, mifepristone, 11(-(4-dimethylaminoethoxyphenyl)-17(-propynyl-17(-hydroxy-4,9-estradien-3one; 17(-hydroxy-17(-19-(4-methylphenyl)androsta-4,9(11)-dien-3-one; benzaldehyde, 4-[(11 beta, 17beta)-17-methoxy-17-(methoxymethyl)-3-oxoestra-4, 9-dien-11beta-yl]-, 1-oxime or other 11beta-benzaldoxime-substituted SPRMs; 5-(7-fluoro-3,3-dimethyl-2-oxo-2,3-dihydro-1H-indol-5-yl)-1-methyl-1H-pyrrole-2-carbonitrile 27.

Other progesterone receptor antagonists which can be employed in the present methods include, but are not limited to, 11-(4-Acetyl-phenyl)-17-hydroxy-13-methyl-17-(1,1,2,2,2-pentafluoro-ethyl)-1,2,6,7,8,11,12,13,14,15,16,17-dodecahydro-cyclopenta [a] phenanthren-3-one, available under the designation “ZK230211”; “ZK98774”; “ZK137316”; 11-(4-Acetyl-phenyl)-17-hydroxy-13-methyl-17-prop-1-ynyl -1,2,6,7,8,11,12,13,14,15,16,17-dodecahydro-cyclopenta [a] phenanthren-3-one, available under the designation “ZK112993”; 4-(17-Methoxy-17-methoxymethyl-13-methyl-3-oxo-2,3, 6,7, 8,11,12,13,14,15,16,17-dodecahydro-1H-cyclopenta [a] phenanthren-11-yl)-benzaldehyde oxime, available under the designation“J867”; 13-Methyl-7, 8,9,11,12,13,14,15,16,17-decahydro-6H-cyclopenta [a] phenanthrene-3, 17-diol, available under the designation “J956”; 3-Fluoro-5-(2,2,4-trimethyl-1, 2-dihydro-quinolin-6-yl)-benzonitrile, available under the designation “LG-120830”; 1,2-dihydro-2,2,4-trimethyl-6-phenlyquinoline, available under the designation “LGO01447”; “LG120753”, “ORG33628” 11-(4-Dimethylamino-phenyl)-17-hydroxy-17-(3-hydroxy-propyl)-13-methyl-1,2,6,7,8,11,12,13,14,15,16,17-dodecahydro-cyclopenta [a] phenanthren-3-one, available under the designation “ZK-98299” or “onapristone”; lilopristone (11-(4-Dimethylamino-phenyl)-17-hydroxy-17-(3-hydroxy-propenyl) -13-methyl-1,2,6,7,8,11,12,13,14,15,16,17-dodecahydro-cyclopenta [a] phenanthren-3-one), and salts and derivatives or analogs of these compounds which function as a progesterone receptor antagonist.

Other steroidal progesterone receptor antagonists include metabolites of mifepristone, e.g. monodemethylated mifepristone, didemethylated mifepristone, and 17-α-[3′-hydroxy-propynyl] mifepristone (as described in Attardi et al, Journal of Steroid Biochemistry & Molecular Biology, 2004, 88: 277-288).

In a most preferred embodiment, the steroidal progesterone receptor antagonist is mifepristone.

The compounds may be in the form of pharmaceutically acceptable salts, esters, optically active isomers, racemates or hydrates.

Dosage and Regimen:

Lubiprostone is formulated in association with a pharmaceutically acceptable carrier. Lubiprostone and the progesterone receptor antagonist may be administered separately, preferably sequentially and, if desired, by different routes.

The active ingredients may be administered by any convenient route, including oral, sublingual, buccal, parenteral, transdermal, vaginal, intra-uterine, rectal, nasal, etc. Preferably the pharmaceutical composition is suitable for oral or parenteral administration. In a particular embodiment, the composition is suitable for intradermal administration. For instance, the composition may be in the form of a patch or an implant. In still another embodiment, the composition is suitable for intra-uterine administration. In still another embodiment, the composition is suitable for vaginal administration.

In a particular embodiment, lubiprostone is administered 24 to 48 hours after a progesterone receptor antagonist. In a particular protocol, lubiprostone may be administered at least once or twice a day, during one or two days. In a specific embodiment, lubiprostone is administered orally once or twice a day, e.g. at about 8 μg twice a day, during two days, about 24 hours after a progesterone receptor antagonist, such as mifepristone.

In a particular embodiment the progesterone receptor antagonist may be administered orally, as well as lubiprostone. In another embodiment, the progesterone receptor antagonist and lubiprostone are administered within a single pharmaceutical composition, preferably orally.

In a particular embodiment, lubiprostone may be administered at a daily dosage of 8 to 48 μg, preferably 16 to 32 μg. In a specific example, it is thus provided pharmaceutical compositions comprising from 8 to 48 μg lubiprostone.

The progesterone receptor antagonist may be administered at a daily dosage of 50 to 800 mg, preferably 200 to 600 mg. In particular, it is thus provided pharmaceutical compositions or kits comprising lubiprostone and from 50 to 800 mg of a progesterone receptor antagonist.

For separate administrations, a kit may be convenient. Such kit comprises
a. a pharmaceutical composition comprising lubiprostone, or a salt or ester thereof.
b. another pharmaceutical composition comprising a progesterone receptor antagonist, such as mifepristone.

The pharmaceutical compositions respectively comprise a pharmaceutically acceptable carrier. In a preferred embodiment, both pharmaceutical compositions are in the form of tablets. In a specific embodiment, the kit is a blister comprising the tablets of active ingredients in a pharmaceutically effective amount for achieving one pregnancy interruption or menstrual induction. The kit may further comprise a leaflet or labelling specifying the medical indication, for pregnancy interruption or menstrual induction.

Routes of Administration:

For a brief review of present methods for drug delivery, see, Langer, Science 249:1527-1533 (1990), which is incorporated herein by reference. Methods for preparing administrable compounds are known or are apparent to those skilled in the art and are described in more detail in, for example, Remington's Pharmaceutical Science, 17th ed., Mack Publishing Company, Easton, Pa. (1985), which is incorporated herein by reference, and which is hereinafter referred to as “Remington.”

For solid compositions, conventional non toxic solid carriers may be used which include, for example, pharmaceutical grades of mannitol, lactose, starch, magnesium stearate, sodium saccharine, talcum, cellulose, glucose, sucrose, magnesium, carbonate, and the like. For oral administration, a pharmaceutically acceptable nontoxic composition is formed by incorporating any of the normally employed excipients, such as those carriers previously listed.

Oral solid dosage forms are preferentially compressed tablets or capsules. Compressed tablets may contain any of the excipients described above which are diluents to increase the bulk of the active ingredient so that production of a compressed tablet of practical size is possible. Binders, which are agents which impart cohesive qualities to powdered materials are also necessary. Starch, gelatine, sugars such as lactose or dextrose, and natural and synthetic gums are used. Disintegrants are necessary in the tablets to facilitate break-up of the tablet. Disintegrants include starches, clays, celluloses, algins, gums and crosslinked polymers. Lastly small amounts of materials known as lubricants and glidants are included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants. Procedures for the production and manufacture of compressed tablets are well known by those skilled in the art (See Remington).

Capsules are solid dosage forms using preferentially either a hard or soft gelatine shell as a container for the mixture of the active ingredient and inert ingredients. Procedures for production and manufacture of hard gelatin and soft elastic capsules are well known in the art (See Remington).

Buccal forms or devices are also useful, such as those described in U.S. patent application 20050208129, herein incorporated by reference. U.S. patent application 20050208129 describes a prolonged release bioadhesive mucosal therapeutic system containing at least one active principle, with an active principle dissolution test of more than 70% over 8 hours and to a method for its preparation. Said bioadhesive therapeutic system comprises quantities of natural proteins representing at least 50% by weight of active principle and at least 20% by weight of said tablet, between 10% and 20% of an hydrophilic polymer, and compression excipients, and comprising between 4% and 10% of an alkali metal alkylsulphate to reinforce the local availability of active principle and between 0.1% and 1% of a monohydrate sugar.

For parenteral administration, fluid unit dosage forms are prepared utilizing the compounds and a sterile vehicle, water being preferred. The active ingredient, depending on the vehicle and concentration used, can be either suspended or dissolved in the vehicle. In preparing solutions the compound can be dissolved in water for injection and filtered sterilized before filling into a suitable vial or ampoule and sealing. Advantageously, adjuvants such as a local anesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection is supplied to reconstitute the liquid prior to use. Parenteral suspensions can be prepared in substantially the same manner except that the compounds are suspended in the vehicle instead of being dissolved and sterilization cannot be accomplished by filtration. The compound can be sterilized by exposure to ethylene oxide before suspending in the sterile vehicle. Advantageously, a surfactant or wetting agent is included in the composition to facilitate uniform distribution of the active ingredient.

Additionally, a suppository can be employed to deliver the active ingredient. The active compound can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate. These suppositories can weigh from about 1 to 2.5 mg.

Transdermal delivery systems comprising a penetration enhancer and an occlusive backing are of use to deliver the active ingredient. Examples of penetration enhancers include dimethyl sulfoxide, dimethyl acetamide and dimethylformamide.

Systems comprising polymeric devices which slowly release or slowly erode and release within the body to provide continuous supplies of the active ingredient are also of use.

The below example illustrates the invention without limiting its scope.

### EXAMPLE

Twelve women requesting early pregnancy termination (<64 days of amenorrhea) have received 200 mg mifepristone once, orally followed 24 hours later by the oral administration of lubiprostone (8 μg twice daily for 2 consecutive days). Efficacy rate (i.e. complete pregnancy termination, without need for surgical procedure) was 97 percent. In one woman, ovular fragments had to be removed at the uterine os with a forceps. Median bleeding time was 8 days. There was no specific adverse event except mild diarrhea in 3 subjects.

1. A method for terminating pregnancy or menstrual induction, which method comprises administering a female subject, with lubiprostone, or a salt or ester thereof.

2. The method of claim 1, which method further comprises administering a progesterone receptor antagonist.

3. The method of claim 2, wherein the progesterone receptor antagonist is a steroidal progesterone receptor antagonist.

4. The method of claim 3, wherein the steroidal progesterone receptor antagonist is selected from the group consisting of mifepristone, monodemethylated mifepristone, didemethylated mifepristone, and 17-α-[3′-hydroxy-propynyl] mifepristone.

5. The method of claim 2, wherein the progesterone receptor antagonist is a non-steroidal glucocorticoid receptor antagonist.

6. The method of any of claim 2, wherein the progesterone receptor antagonist and lubiprostone, or a salt or ester thereof, are administered separately.

7. The method of claim 2, wherein the progesterone receptor antagonist and lubiprostone, or a salt or ester thereof, are administered within a single pharmaceutical composition.

8. The method of claim 1, wherein lubiprostone, or a salt or ester thereof, is administered orally.

9. The method of claim 1, wherein lubiprostone, or a salt or ester thereof, is administered by vaginal route.

10. The method of claim 1, wherein lubiprostone, or a salt or ester thereof, is administered at a daily dosage of 8 to 48 μg.

11. The method of claim 2, wherein the progesterone receptor antagonist is administered at a daily dosage of 50 to 800 mg.

12. The method of claim 1, wherein the subject is a human female.

13. The method of claim 12, wherein the subject is a woman who is less than three month pregnant.

14. A pharmaceutical composition comprising lubiprostone, or a salt or ester thereof, and a progesterone receptor antagonist, in association with a pharmaceutically acceptable carrier.

15. A kit comprising:
a. a pharmaceutical composition comprising lubiprostone, or a salt or ester thereof,
b. another pharmaceutical composition comprising a progesterone receptor antagonist, such as mifepristone.

16. (canceled)
